(19) United States
(12) Patent Application Publication
(10) Pub. No.: US 2005/0222542 A1
(43) Pub. Date: Oct. 6, 2005

(54) Antimicrobial delivery system and application device

(76) Inventors: Jonathan Karl Burkholz, Salt Lake City, UT (US); Minh Quang Hoang, Sandy, UT (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 10/818,192

(22) Filed: Apr. 5, 2004

(51) Int. Cl. A61M 35/00

(52) U.S. Cl. 604/289; 604/3

(57) ABSTRACT

An antimicrobial solution delivery system comprises a first reservoir containing an antimicrobial solution connected to a second reservoir housing an application member. The system is operable in a first inactivated condition, a second activated condition, and a third application condition. In the first condition, the first reservoir is sealed with respect to the second reservoir such that the solution does not flow from the first reservoir to the second reservoir. In the second condition, the solution may flow from the first reservoir to the second reservoir. In the third condition at least a portion of the application member is exposed to deliver the antimicrobial solution.

### FIELD OF THE INVENTION

The present invention is directed to an antimicrobial delivery system and application device containing a dose of antimicrobial solution and an applicator member.

### BACKGROUND OF THE INVENTION

Many medical procedures involve the use of a percutaneous catheter or intravenous (I.V.) catheters inserted into the body of a patient for fluid infusion or removal. Often, the catheter remains in place for more than 48 hours. During this period, the catheter needs to be reliably positioned and accessible for attachment and detachment of fluid handling devices. In addition to the need to keep a catheter on the patient's skin, the placement site needs to be protected from microorganisms to prevent catheter-related blood stream infection.

Because, in many cases, the catheter may remain in place for 24 to 72 hours or more, development of infections by common skin microorganisms at the penetration site are common. As a result, practitioners routinely inspect the penetration site for evidence of infection. Because examination of the actual penetration site is sometimes difficult when adhesive tape is used for securing the catheter, there is a need for a catheter that is pre-coated with an antimicrobial solution before insertion to protect the catheter from colonization and suppress growth of microorganisms around the penetration site. Additionally, if the penetration site is aseptic by coating the skin surface with an antimicrobial film, a further benefit would be realized.

Recent reports indicating an increasing rate of infection involving needleless access systems, especially in home-care settings, have emphasized the need for medical devices that disinfect the I.V. access systems. Without using such a device, the I.V. access system is at high risk of microbial contamination through the transfer of microorganisms into the fluid path. Typically, contaminated I.V. fluid produces clinical illness in patients possessing intact immune systems. Moreover, the risk of clinical illness becomes more significant in immuno-compromised patients and may result in blood stream infection or morbidity.

Alcohol has long been used as a disinfectant and has been recognized for its broad spectrum, fast-acting antimicrobial effectiveness. It has, therefore, been widely used as the active ingredient in numerous products for cleaning solutions for medical devices, I.V. sites, surgical skin preparation and skin disinfection. Alcohol-based solutions used as a surgical preparation have many advantages over soap solutions and water-based antiseptic solutions, including reduced prepping and solution drying time. A variety of means for applying these alcohol based solutions has been used including towelettes, swab sticks, foam sponge applicators, etc.

A need still exists, however, for a safe and convenient handheld antimicrobial delivery system that can be used for multiple applications (i.e., skin/site prep, catheter coating and I.V. access cleaning device).

### SUMMARY OF THE INVENTION

The present invention is directed to an antimicrobial solution delivery system comprising a first reservoir containing an antimicrobial solution connected to a second reservoir housing an application member. The system is operable in a first inactivated condition, a second activated condition, and a third application condition. In the first condition, the first reservoir is sealed with respect to the second reservoir such that the antimicrobial solution does not flow from the first reservoir to the second reservoir. In the second condition, the antimicrobial solution may flow from the first reservoir to the second reservoir. In the third condition, at least a portion of the application member is exposed to deliver the antimicrobial solution.

In one embodiment, the application member is an absorbent member configured to absorb the antimicrobial solution. In another embodiment, a rupturable seal can be positioned between the first reservoir and the second reservoir to prevent the antimicrobial solution from flowing from the first reservoir into the second reservoir in the inactivated condition.

In another embodiment, the system comprises a package having a body portion sealed with a lid portion, and the first and second reservoirs are formed in the body portion. In the activated condition, antimicrobial solution may flow from the first reservoir to the second reservoir and into contact with the application member. The system may be moved from the inactivated condition to the activated condition by applying pressure to the first reservoir to force the solution contained therein toward the second reservoir. The pressure may break the seal between the first reservoir and the second reservoir. In one embodiment, the amount of solution that flows into the second reservoir may be controlled by varying the pressure applied to the first reservoir.

The system may be moved into the application condition by exposing at least a portion of the application member. In one embodiment, the second reservoir has a wall having at least one pre-defined rupturable portion. The rupturable portion can comprise a perforated wall section. In another embodiment, the rupturable portion may comprise a crease area extending across the second reservoir, and the second reservoir may be bent at the crease area to rupture the second reservoir and expose the application member. In one embodiment, the crease extends perpendicular to the longitudinal axis of the package. The package lid may include notches adjacent the crease section to facilitate bending of the package. According to one aspect of the present invention, the package may be used to apply the antimicrobial solution to a cleaning site without directly contacting the exposed application member.

In another embodiment, the wall of the second reservoir can have a plurality of rupturable portions, and the application member may have a pre-formed slit formed therein. The slit may be positioned adjacent the crease section and, when the device is in the application condition, the slit may be exposed to form a V-groove. The V-groove may be used to apply antimicrobial solution to a cylindrical device.

In another embodiment, the perforated wall section defines the perimeter of a peelable wall portion. The peelable wall portion can be peeled away from the second reservoir to expose at least a portion of application member. In another embodiment, the system may include a crease and a peelable portion for exposing at least a portion of the application member. In yet another embodiment, the absorbent member may comprise a pad made of a non-woven material, and the antimicrobial solution may comprise 70% alcohol, 2% CHG, 5% moisturizers and 23% USP water.

### BRIEF DESCRIPTION OF THE DRAWINGS

FIG. 1 is a side view of a first embodiment of an antimicrobial delivery system constructed according to the present invention;

FIG. 2 is a top plan view of the embodiment of FIG. 1;

FIG. 3 is a cross sectional view of the embodiment of FIG. 1 prior to activation taken along lines 3-3 of FIG. 2;

FIG. 4 is a cross sectional view of the embodiment of FIG. 1 after activation taken along lines 3-3 of FIG. 2;

FIG. 5 is a side view of the embodiment of FIG. 1 in the application condition;

FIG. 6 is a side view of another embodiment of an antimicrobial delivery system constructed according to the present invention shown in the application condition;

FIG. 7 is a top plan view of another embodiment of an antimicrobial delivery system constructed according to the present invention;

FIG. 8 is a cross sectional view of the embodiment of FIG. 7 shown in the application condition taken along lines 8-8 of FIG. 7;

FIG. 9 is a perspective view a top plan view of another embodiment of an antimicrobial delivery system constructed according to the present invention; and

FIG. 10 is a side view of the embodiment of FIG. 9 shown in the application condition.

### DETAILED DESCRIPTION OF THE INVENTION

The invention disclosed herein relates generally to a convenient hand-held integrated antimicrobial delivery system and an application device or kit for cleaning and disinfecting skin/site preparation, preparation of a point-of-use catheter, and/or to clean catheter access devices. With respect to site preparation, the device can be used as a skin applicator to disinfect a patient's skin prior to insertion of a catheter. The device can also be used, for example, for cleaning and/or antimicrobial coating of I.V. catheter tubing before insertion or as an I.V. access device disinfectant.

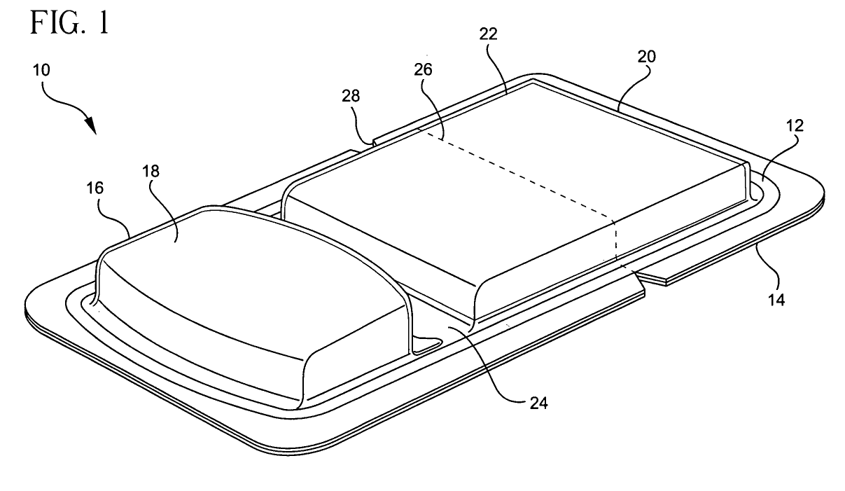

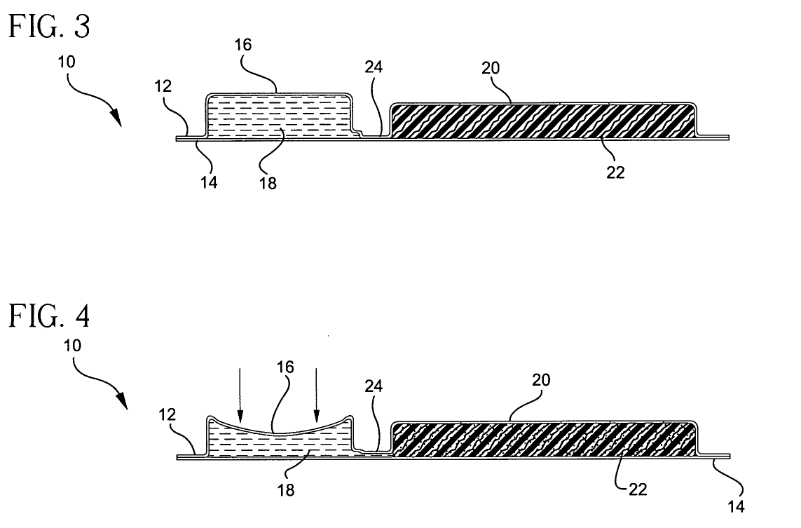

Referring to the drawings in which like reference characters refer to like parts throughout the several views thereof, FIGS. 1-4 depict a first preferred embodiment of an antimicrobial delivery system and application device 10 constructed in accordance with the present invention. Applicator 10 generally comprises a package with a formed poly body 12 sealed with a lid 14. In one preferred embodiment, poly body 12 can be made from a plastic material and lid 14 may be made from a foil material, as is commonly known in the blister pack art. In general, formed body 12 contains a chamber, web or reservoir 16 to hold a liquid antimicrobial solution 18 and an adjacent chamber, web, or reservoir 20 to hold or house an application member or absorbent member 22. Initially, in an inactive condition, a seal area 24 positioned between reservoir 16 and reservoir 20 prevents the antimicrobial solution 18 from entering into reservoir 20. Advantageously, the device may be manufactured such that solution 18 and absorbent member 22 are packaged aseptic in the inactive condition. As can be seen in the cross-sectional view of FIG. 3, showing device 10 prior to activation, in this embodiment seal 24 comprises a membrane region where body 12 and lid 14 meet. In this regard, the seal area 24 between the solution and absorbent reservoirs 16, 20 is generally weaker than the seals around the perimeter of the poly body 12 and lid 14.

As best seen in FIG. 4, showing device 10 after activation, in operation, device 10 can be moved into an active condition to permit the flow of antimicrobial solution 18 from reservoir 16 to reservoir 20 and into contact with absorbent member 22. In particular, the device may be activated by applying pressure to solution-containing chamber 16 to force the solution 18 toward reservoir 20. This pressure may break the seal 24 between the solution reservoir 16 and absorbent reservoir 20, allowing solution to flow into the absorbent member 22. Once seal 24 is broken, the amount of solution flow into reservoir 20 and the degree of liquid saturation of absorbent member 22 may be controlled by varying the pressure applied to chamber 16.

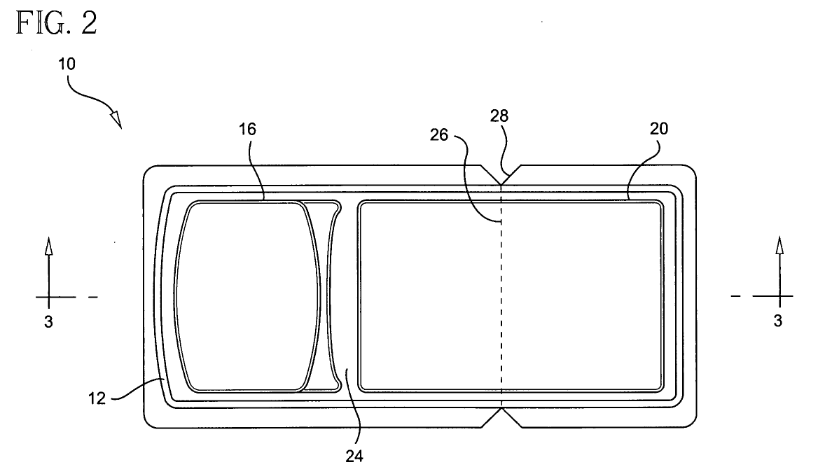

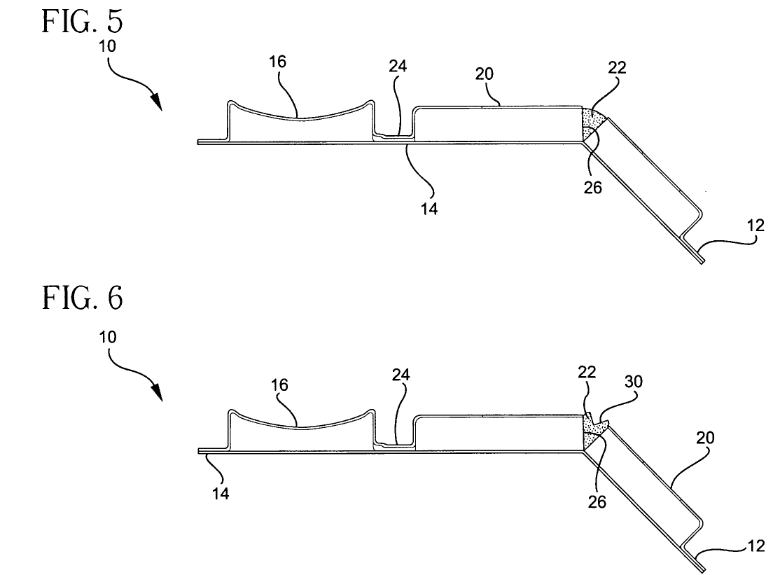

Referring to FIG. 5, when the absorbent member 22 is saturated with solution to a desired amount, applicator 10 can be further moved into an application mode or condition by exposing at least a portion of wetted absorbent member 22 beyond body 12 for skin, catheter or device cleaning. For example, with respect to the embodiment depicted in FIGS. 1-4, reservoir 20 of body 12 may have one or more pre-defined breakable features, perforations or creases 26 to provide access to absorbent member 22 and permit application of the antimicrobial solution at a desired application site. As best seen in FIG. 2, in this embodiment body 12 has a crease 26 positioned across the midsection of reservoir 20. Crease 26 generally runs perpendicular to the longitudinal axis of applicator 10 and is formed in the body 12 as opposed to lid 14. Lid 14 may also preferably include notches 28 adjacent the point at which creases 26 meet lid 14. In general, notches 28 may facilitate the bending of body 12 at crease 26. As best seen in FIG. 5, body 12 may be bent backwards at the crease area 26, thereby breaking reservoir 20 at crease 26 and exposing the wetted pad for skin, catheter or device cleaning. As body 12 is bent further backwards, a greater portion of the absorbent member 22 is exposed for use such that the exposed surface of the absorbent member may be used without the edges of the crease area 26 contacting or interfering with the skin or application site during cleaning. In this regard, device 10 may be used by a practitioner as an applicator tool to deliver antimicrobial solution to a cleaning site without the need to directly contact the wetted absorbent member 22 or the application site and advantageously maintaining the site as aseptic or sterile as possible. It will be appreciated that such a feature is advantageous for practitioners to maintain the application site as sterile as possible. Also, such a feature may be particularly advantageous when dealing with highly contagious diseases transmitted by blood. Furthermore, it will be appreciated that as a result the applicator 10 may be conveniently and cleanly disposed of after use without a significant mess. In alternative embodiments, more than one crease 26 may be provided in reservoir 20. In this regard, a practitioner may be able to use the same applicator to clean and/or disinfect multiple sites with the same device.

Referring to FIG. 6, in another aspect of the present invention, absorbent member 22 may have a pre-formed slit or notch 30 formed therein and positioned adjacent crease 26. In operation, applicator 10 may be moved into the application condition by bending package body 12 backward about crease 26 to break package body 12 along crease 26 to expose a portion of wetted absorbent member 22. In this regard, notch 30 may be exposed from package body 12 and form a V-shaped groove to perform, for example, a cleaning and/or coating function of a catheter. The V-shaped groove shape may be particularly well suited for cleaning and/or disinfecting cylindrical medical devices such as catheter tubing, needles, etc., especially for cleaning and or coating a catheter. For catheter cleaning, the portion of the catheter to be cleaned may be placed in the V-shaped groove and drawn through, thereby applying antimicrobial solution to the catheter surface. As body 12 may be bent further backwards, the V-shaped groove may become wider, thereby exposing a greater portion of the absorbent member 22 such that the V-shaped groove may be used with various sizes of tubing, needles and/or catheters. As described above, device 10 may be used by a practitioner as an applicator tool to deliver antimicrobial solution to the cleaning site without the need to directly contact the wetted absorbent member 22 or the application site and can advantageously maintain, for example, a catheter as aseptic or sterile as possible. In an alternative embodiment, slit or notch 30 may be cut into absorbent member by a practitioner after the absorbent member 22 is exposed from reservoir 20 to create a V-shaped groove which may then function in a similar manner as described above.

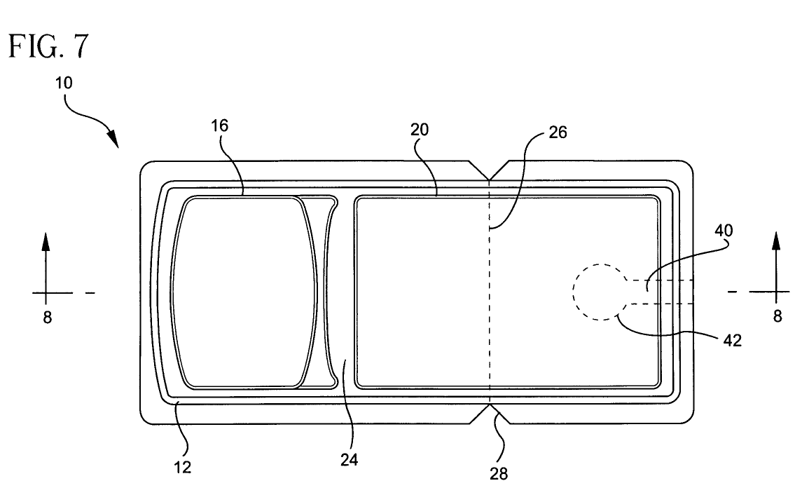

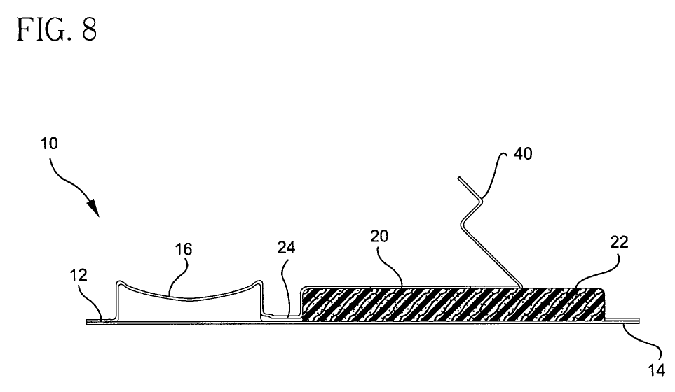

Referring to FIGS. 7 and 8, in another aspect of the present invention, applicator 10 can include an alternative application mode or condition comprising a peel back feature for exposing at least a portion of wetted absorbent member 22. For example, reservoir 20 of body 12 may have one or more pre-defined peel back portions 40 having a perimeter 42 that is perforated or may be breakable such that portion 40 may be peeled back to provide access to absorbent member 22 and permit application of the antimicrobial solution at a desired application site. As best seen in FIG. 7, in this embodiment body 12 has a peel back portion 40 with a key hole shaped perimeter 42 positioned adjacent an edge of reservoir 20. It will be appreciated that in alternate embodiments, peel back portion 40 may have varied shapes and dimensions as desired. Perimeter 42 is generally perforated through reservoir 20 as opposed to lid 14 and portion 40 of body 12 may peeled forward, as shown in FIG. 8, by breaking reservoir 20 along perimeter 42 to expose the wetted pad for skin, catheter or device cleaning. The peel back portion 40 is generally well suited for cleaning and/or disinfecting large or oddly shaped devices or application sites that may be more difficult to clean using the fold back crease 26 and method described above. In particular, peel back portion 40 may be advantageously used to clean and/or disinfect I.V. access devices such as needleless valves, among other things. As with prior embodiments, device 10 may be used by a practitioner as an applicator tool to deliver antimicrobial solution to such devices without the need to directly contact the wetted absorbent member 22 or the application site and facilitates maintaining the application site as sterile or aseptic as possible.

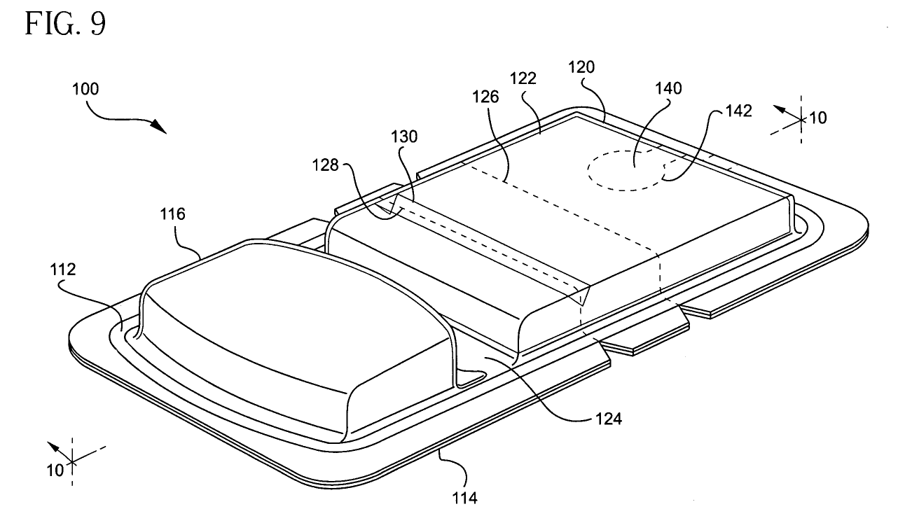

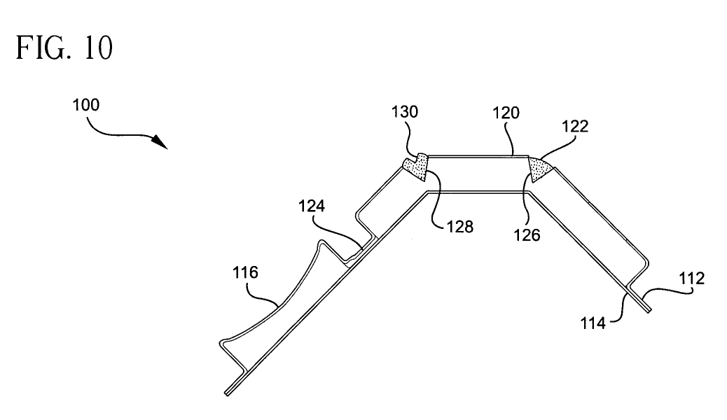

Referring to FIGS. 9 and 10, another embodiment of an antimicrobial delivery system and application device 100 according to the present invention is shown. In this embodiment, applicator 100 includes multiple application modes or conditions for exposing absorbent member 122. For example, reservoir 120 of body 112 comprises a crease 126, a crease 128 adjacent a notch 130 in absorbent member 122 and a peel back feature 140 for exposing at least a portion of wetted absorbent member 122 in operation. As shown in FIG. 9, it will be appreciated that a practitioner may be able to use a single applicator for different purposes or multiple purposes, depending on the type of site or device to be cleaned and/or disinfected. For example, device 100 may be used first to clean and/or disinfect a skin site utilizing the exposed region of absorbent member 122 adjacent crease 126, then device 100 may be used to clean a catheter, tubing, etc. utilizing the exposed V-shaped groove 130 adjacent crease 128, and then peel back feature 140 may be used to clean and/or disinfect, for example, an I.V. access device.

In general, reservoirs 16, 20 can have various shapes and dimensions such as, for example, rectangular, cylindrical, polygonal etc., and can be incorporated into any type of package. In a preferred embodiment, reservoir 16 is sized and dimensioned to contain an amount of solution sufficient to completely moisten the absorbent member 22 so that the absorbent member 22 contains enough solution to clean and or disinfect as desired. In a preferred embodiment, reservoir 16 is sized and dimensioned to contain from about 0.5 mL to about 10 mL of solution. Reservoir 20 can also have various shapes and dimensions to accommodate virtually any shape or size absorbent member 22. In other embodiments, reservoir 20 can also have alternative means of exposing absorbent member 22 for application, such as a rupturable membrane, a puncturable type material, a mechanical opening means, etc. Also, applicator device 10 may have various overall shapes in alternative embodiments. For example, in one alternative embodiment, applicator device 10 may have a butterfly shape to facilitate bending backward to expose absorbent member 22.

In general, absorbent member 22 may be made from any material suitable for absorbing liquid. In one preferred embodiment, absorbent member 22 comprises a pad made of a non-woven material, such as a foam or sponge. In another embodiment, absorbent member 22 comprises a towelette made of a non-woven material.

The solution contained in the device is an alcohol-based antimicrobial solution (60-95%), which may contain one or more additional antimicrobial agents such as CHG, PCMX, triclosan, octenidine, hexachlorophene, PVP-I, iodine, quaternary compounds and the like in the range of 0.05% to 5% w/w. The alcohol can be, for example, ethyl, isopropal, n-propanol or a mixture of alcohols. The solution may also contain one or more additional components such as dimethicone, glycerin, cationic polymer such as PVP, cellulose, docosanol, BTMS, behenyl alcohol, poloxamer and the like.

The solution can be a “rinseless” or a “rinse” type of solution. One preferred solution is a rinseless antimicrobial solution containing 70% alcohol, 2% CHG, 5% moisturizers and 23% USP water. Other ingredients may be added as mentioned above.

While this invention is satisfied by embodiments in many different forms, as described in detail preferred embodiments of the invention, with the understanding that the present disclosure is to be considered as exemplary of the principles of the invention and is not intended to limit the invention to the embodiments illustrated and described. Numerous variations may be made by persons skilled in the art without departure from the spirit of the invention. The scope of the invention will be measured by the appended claims and their equivalents.

1. An antimicrobial solution delivery system, comprising:
a first reservoir containing an antimicrobial solution; and
a second reservoir connected to the first reservoir, the second reservoir housing an application member,
the system operable in a first inactivated condition, a second activated condition, and a third application condition;
wherein in the first condition the first reservoir is sealed with respect to the second reservoir such that the solution cannot flow from the first reservoir to the second reservoir,
wherein in the second condition the solution may flow from the first reservoir to the second reservoir, and
wherein in the third condition at least a portion of the application member is exposed to deliver the antimicrobial solution.

2. The antimicrobial solution delivery system of claim 1, wherein the application member is an absorbent member configured to absorb the antimicrobial solution.

3. The antimicrobial solution delivery system of claim 1, wherein a rupturable seal is positioned between the first reservoir and the second reservoir to prevent the antimicrobial solution from flowing from the first reservoir into the second reservoir in the inactivated condition.

4. The antimicrobial solution delivery system of claim 3, wherein the seal comprises a rupturable membrane region.

5. The antimicrobial solution delivery system of claim 3, wherein the system comprises a package having a body portion sealed with a lid portion, and wherein the first and second reservoirs are formed in the body portion and the seal comprises a portion of the sealed lid between the first and second reservoirs.

6. The antimicrobial solution delivery system of claim 1, wherein in the activated condition antimicrobial solution may flow from the first reservoir to the second reservoir and into contact with the application member.

7. The antimicrobial solution delivery system of claim 1, wherein the system may be moved from the inactivated condition to the activated condition by applying pressure to the first reservoir to force the solution contained therein toward the second reservoir.

8. The antimicrobial solution delivery system of claim 7, wherein the pressure may break the seal between the first reservoir and the second reservoir.

9. The antimicrobial solution delivery system of claim 7, wherein in the activated condition the amount of solution that flows into the second reservoir may be controlled by varying the pressure applied to the first reservoir.

10. The antimicrobial solution delivery system of claim 1, wherein the system may be moved into the application condition by exposing at least a portion of the application member.

11. The antimicrobial solution delivery system of claim 1, wherein the second reservoir has a wall having at least one predefined rupturable portion.

12. The antimicrobial solution delivery system of claim 11, wherein the rupturable portion comprises a perforated wall section.

13. The antimicrobial solution delivery system of claim 11, wherein the rupturable portion comprises a crease area extending across the second reservoir, and the second reservoir may be bent at the crease area to rupture the second reservoir and expose the application member.

14. The antimicrobial solution delivery system of claim 1, wherein the crease extends perpendicular to the longitudinal axis of the package.

15. The antimicrobial solution delivery system of claim 5, wherein the second reservoir may have a wall having at least one pre-defined rupturable crease section extending across the second reservoir, and the package may be bent at the crease section to rupture the second reservoir and expose the application member, and wherein the lid includes notches adjacent the crease section to facilitate bending of the package.

16. The antimicrobial solution delivery system of claim 15, wherein when a greater portion of the application member is exposed, the more the package is bent.

17. The antimicrobial solution delivery system of claim 15, wherein the package may be used to apply the antimicrobial solution to a cleaning site without directly contacting the exposed application member.

18. The antimicrobial solution delivery system of claim 11, wherein the wall of the second reservoir has a plurality of rupturable portions.

19. The antimicrobial solution delivery system of claim 13, wherein the application member has a pre-formed slit formed therein.

20. The antimicrobial solution delivery system of claim 19, wherein the slit is positioned adjacent the crease section and when the device is in the application condition the slit may be exposed to form a V-shaped groove.

21. The antimicrobial solution delivery system of claim 20, wherein the V-shaped groove may be used to apply antimicrobial solution to a cylindrical device.

22. The antimicrobial solution delivery system of claim 12, wherein the perforated wall section defines the perimeter of a peelable wall portion and the peelable wall portion can be peeled away from the second reservoir to expose at least a portion of the application member.

23. The antimicrobial solution delivery system of claim 18, wherein the second reservoir comprises:
at least one rupturable portion comprising a crease area extending across the second reservoir, and the second reservoir may be bent at the crease area to rupture the second reservoir and expose the application member; and
a perforated wall section defining the perimeter of a peelable wall portion and the peelable wall portion can be peeled away from the second reservoir to expose at least a portion of the application member.

24. The antimicrobial solution delivery system of claim 1, wherein the first reservoir is sized and dimensioned to contain from about 0.5 to about 10 mL of solution.

25. The antimicrobial solution delivery system of claim 2, wherein the absorbent member comprises a pad made of a non-woven material.

26. The antimicrobial solution delivery system of claim 25, wherein the pad is made of a foam material.

27. The antimicrobial solution delivery system of claim 25, wherein the pad is a sponge.

28. The antimicrobial solution delivery system of claim 2, wherein the absorbent member comprises a towelette made of a non-woven material.

29. The antimicrobial solution delivery system of claim 1, wherein the antimicrobial solution is a solution containing 70% alcohol, 2% CHG, 5% moisturizers and 23% USP water.

30. An antimicrobial solution application kit, comprising:
a main body package having a first reservoir and a second reservoir, the first reservoir containing an antimicrobial solution, and the second reservoir housing an absorbent member,
wherein the package is moveable between a first inactivated position, a second activated position, and a third application position;
wherein in the first position the solution does not flow from the first reservoir to the second reservoir,
wherein in the second position the solution may flow from the first reservoir to the second reservoir, and
wherein in the third position at least a portion of the absorbent member is exposed form the second reservoir to deliver the antimicrobial solution.

31. The kit of claim 30, wherein the device may be moved from the inactivated condition to the activated condition by applying pressure to first reservoir to force the solution contained therein toward the second reservoir.

32. The kit of claim 30, wherein in the activated condition the amount of solution that flows into the second reservoir may be controlled by varying the pressure applied to the first reservoir.

33. The kit of claim 30, wherein the second reservoir has a wall having at least one pre-defined rupturable portion.

34. The kit of claim 33, wherein the rupturable portion comprises a perforated wall section.

35. The kit of claim 33, wherein the rupturable portion comprises a crease area extending across the second reservoir, and the second reservoir may be bent at the crease area to rupture the second reservoir and expose the application member.
